(19) United States
(12) Patent Application Publication
(10) Pub. No.: US 2020/0312442 A1
(43) Pub. Date: Oct. 1, 2020

(54) WASTE MANAGEMENT APPARATUS, SYSTEM, AND METHOD

(71) Applicant: Abbey Garrett LLC, Frisco, TX (US)

(72) Inventors: John Hairr, Frisco, TX (US); Chuen-Shin Wang, Frisco, TX (US)

(21) Appl. No.: 16/372,059

(22) Filed: Apr. 1, 2019

(51) Int. Cl. G16H 40/20

(52) U.S. Cl. CPC G16H 40/20

(57) ABSTRACT

A waste management apparatus, system, and method that includes a receiving unit having at least one anti-siphon valve, a holding section, and an analysis section. An analysis unit coupled to the analysis section, and configured to analyze a fluid for identification, concentration, and amount. At least two control valves fluidly coupled to the receiving unit, and a waste reservoir fluidly coupled to one or more of the at least two control valves. A computing device may be coupled to and control and receive signals of the analysis unit, and the at least two control valves. The receiving unit can have a first side configured for receiving a fluid transport device, the first side being fluidly coupled to a holding section of the receiving unit, and an analysis section of the receiving unit fluidly coupled to the holding section and a second side of the receiving unit.

### BACKGROUND

### Technical Field

The present disclosure relates to waste management. More particularly, and not by way of limitation, the present disclosure is directed to an apparatus, system and method for management of drug and medication waste.

### Description of Related Art

In hospitals, surgical centers, and doctors' offices around the world there is a growing epidemic of drug use and/or abuse by medical professionals. For example, at a hospital in Dallas, Tex., two nurses overdosed on narcotic drugs taken from the hospital. In some cases, these drugs are misappropriated by medical professionals. This misappropriation can occur by medical professional prescribing the drugs to a non-existent patient, prescribing them to a patient but never administers the drugs, or in other case such as surgery the drugs are reported as used in surgery, or wasted after surgery without any verification. For example, a nurse can claim that a narcotic was used in surgery when in fact, an unused portion remained and was taken by the nurse.

Most hospitals have implemented a system to dispense specific drugs for a surgery. A need exists to teach the usage and disposal of these drugs. In surgical operating rooms, once a vial of a drug has been opened or partially administered to a patient, the remaining amount of the drug must be wasted in an approved manner and documented. Many times the documentation is not done properly, or does not match with the drug dispensed requiring medical personal and/or pharmacists to redocument, or backtrack all of the missing information.

It would be advantageous to have an apparatus, system and method for waste management that overcomes the disadvantages of the prior art. The present disclosure provides such an apparatus, system, and method.

### BRIEF SUMMARY

The present disclosure is a waste management system that includes a receiving unit having at least one anti-siphon valve, a holding section, and an analysis section. The waste management system may also have an analysis unit coupled to the analysis section of the receiving unit, and configured to analyze a fluid for identification, concentration, and amount. The waste management system can also have at least two control valves fluidly coupled to the receiving unit, and a waste reservoir fluidly coupled to one or more of the at least two control valves. A computing device of the waste management system may have at least one storage device, at least one memory unit, one or more central processing units, a display, an input/output bus, and at least one user interface device coupled to the input/output bus, and be coupled to the analysis unit, and the at least two control valves.

Thus, in one aspect, the present disclosure is directed to a waste management apparatus. The waste management apparatus having a receiving unit with at least one anti-siphon valve, and having a first side configured for receiving a fluid transport device. The waste management apparatus may also have a holding section of the receiving unit fluidly coupled to the first side of the receiving unit, and an analysis section of the receiving unit fluidly coupled to the holding section and a second side of the receiving unit. An analysis unit coupled to the analysis section of the waste management system may couple with at least one computing device for controlling and receiving signals from the analysis unit and at least two control valves. Additionally, at least two control valves may be fluidly coupled to the analysis section and also a waste reservoir.

In another aspect, the present disclosure is directed to a waste management method. The waste management method can include receiving a fluid via a receiving unit, and transferring the fluid to an analysis section of the receiving unit. The waste management method may also include analyzing the fluid for identification, concentration, and amount with an analysis unit and the analysis unit being electrically coupled to a computing device. The waste management method may direct the fluid to a waste reservoir that is fluidly coupled to the analysis unit.

### BRIEF DESCRIPTION OF THE DRAWINGS

The novel features believed characteristic of the disclosure are set forth in the appended claims. The disclosure itself, however, as well as a preferred mode of use, further objectives and advantages thereof, will be best understood by reference to the following detailed description of illustrative embodiments when read in conjunction with the accompanying drawings, wherein:

FIG. 1A is a system diagram for medication dispensing, and management.

FIG. 1B is a system diagram for medication dispensing, and management.

FIG. 2 is an illustration of a waste management system.

FIG. 3 is an illustration of a waste apparatus enclosure.

FIG. 4A is an illustration of an analysis unit.

FIG. 4B is an illustration of an audit unit.

FIG. 4C is an illustration of a computing device.

FIG. 5 is an illustration of a waste management apparatus.

FIG. 6 is an illustration of a receiving unit.

FIG. 7A is an illustration of a waste management system interface screen.

FIG. 7B is an illustration of a waste management system interface screen.

FIG. 7C is an illustration of a waste management system interface screen.

FIG. 8A is an illustration of a medication identification curve chart.

FIG. 8B is an illustration of a medication concentration curve chart.

FIG. 9A is an illustration of an identification output for Fentanyal.

FIG. 9B is an illustration of an identification output for Kelamine.

FIG. 9C is an illustration of an identification output for Morphine.

FIG. 10A is an illustration of identification output of a sample that includes Fentanyal and Morphine.

FIG. 10B is an illustration of an identification output of a sample with the previously identified sample element removed.

### DETAILED DESCRIPTION

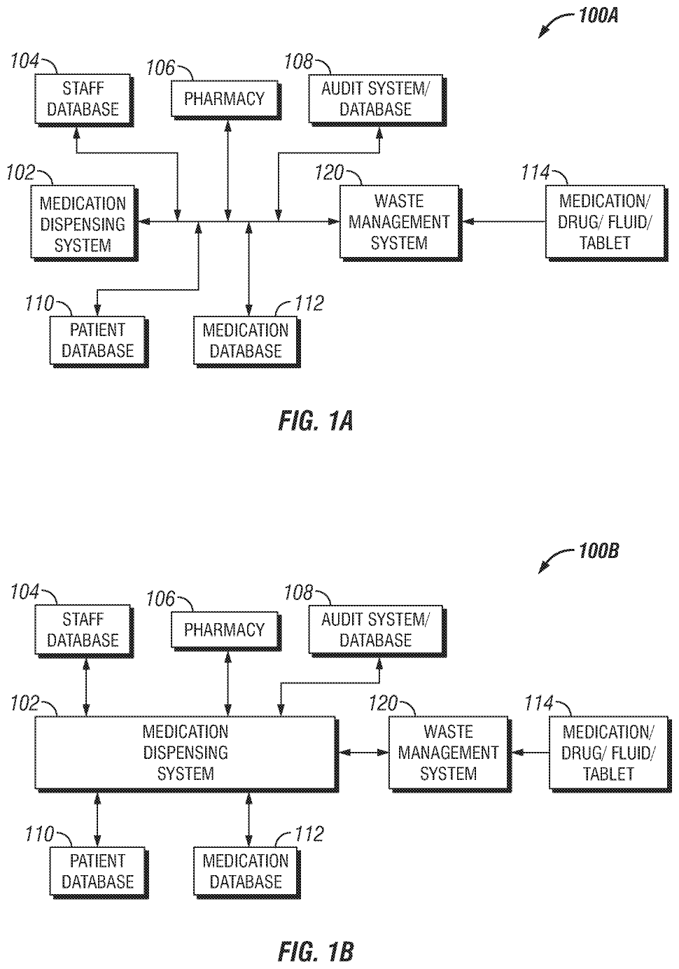

An embodiment of the disclosure will now be described. FIG. 1A is an illustration of Medication Lifecycle 100A. The medication lifecycle 100A can include a Waste Management System (WMS) 120. The waste management system 120 can be coupled and/or linked to a medication dispensing system (MDS) 102. The waste management system 120 can be coupled to a staff or security database 104. A user (not illustrated) may login to the waste management system 120 using password, ID card or badge, ID token or fob, or fingerprint, iris, hand, and/or other biometric identification method. The user (not illustrated) can also access a patient database 110. The patient database 110 can allow a user (not illustrated) to know what medications, doctors, activities, and/or other medical information about a patient. A pharmacy 106 may also be connected to the waste management system 120 and/or medication dispensing system 102. The pharmacy 106, may include or be a database of pharmacy information such as but not limited to, available medications, distributed medications, patient pharmacy records, patient allergy information, related drug or medication information, and/or other information related to medications distributed and/or dispensed in the hospital, clinic, office, or other location. In some embodiments, the pharmacy 106 may be within the same location as the hospital, clinic, office, or other location, while in other embodiments the pharmacy may be at a remote location and accessed through a communication network. The pharmacy 106 may also allow, and/or communicate with a medication database 112. In at least one embodiment, the medication dispensing system 102, and/or the waste management system 120 may also communicate with the medication database 112. The medication database 112 may include information such as, medication identification, alternative names, brand names, generic names, concentrations, versions and/or types of the medication (e.g., pill, or liquid), and/or diluents and/or adulterants. The medication and/or drug 114, can be wasted (e.g., controlled disposal) utilizing the waste management system 102. In at least one embodiment, the medication and/or drug 114 can also include any tablet, powder, and/or fluid to be sampled and/or tested.

FIG. 1B is an illustration of a medication lifecycle 100B. In at least one embodiment of a medication lifecycle 100B, the medication dispensing system 102 can interface with a number of systems, storage, memory, and/or database. For example, the medication dispensing system 102 may interface with a staff and/or security system, and/or database 104. A pharmacy 106, audit system and/or database 108, patient system and/or database 110, and/or medication database 112 may also be connected. A waste management system 120 may be a module that interface and/or connects to the medication dispensing system 102. For example, the waste management system 120 may connect to Pyxis™ medication dispensing system as a module that can be accessed and/or interfaced with by many different portions of the Pyxis system. The module would allow the waste management system to be placed in the same location as a Pyxis or other medication dispensing system. This would allow doctors to waste and dispense in a timely manner, and allow for accountability as these systems have multiple verification and security protocols.

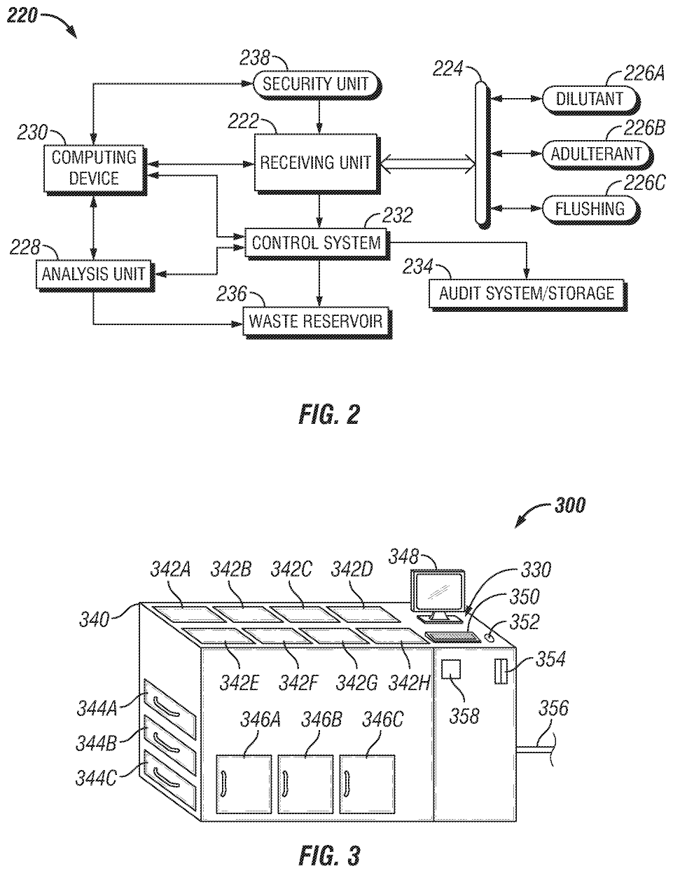

FIG. 2 is an illustration of a waste management system 220. The waste management system 220 can include a security unit 238 for securing and/or preventing unauthorized personnel. In at least one example, the security unit 238 may interface with a security, employee, and/or other databases, or systems. The security unity 238 may also connect and/or interface with the computing device 230. The computing device 230 may allow a user to select a patient and/or a drug previously dispensed by medication dispensing system (not illustrated). The computing device 230 can also be connected to a receiving unit 222, and/or an analysis unit 228, and/or a control system 232. The computing device 230 can have any number of interfacing devices that allow for a user to select, control, and/or engage with the computing device 230 and/or waste management system 220.

For example, the computing device 230 can allow a user to enter the amount of drug and/or medication that will be wasted through waste management system 220. The analysis unit 228 can verify if the amount wasted by a user is correct. In at least one example, the analysis system 228 and/or computing device 230 can communicate to a medication dispensing system (not illustrated) to determine and/or calculate the amount of drug wasted and/or administered, and verify with the user provided information. The user may provide the information through user interfaces with the computing device 230.

The drug and/or medication to be wasted can be received from a user with the receiving unit 222. In at least one embodiment, the drug and/or medication is a fluid. While in some examples the drug and/or medication may also be a tablet, gel, powder, and/or other medically approved material or product. Medically approved materials or products are those approved, under investigation, testing and/or review by the Federal Drug Administration. In other examples, the drug, and/or medication may not be a drug and/or medication but a fluid and/or other sample to be tested for drugs and/or medication such as, but not limited to urine, blood, and/or hair samples.

In at least one example, the receiving unit 222 can allow for a user to connect a syringe with medication to be wasted to the waste management system 220 and request verification of drug identification, amount and concentration from the analysis unit 228 that can be coupled to a fluid control system 232. The receiving unit 222 may be fluidly coupled to the control system 232. In at least one example, receiving unit 222, and/or the control system 232 may individually and/or in combination be coupled to the analysis unit 228. The control system 232 may include a volume mechanism that can transfer the contents of the receiving unit to a waste reservoir and/or an audit system or storage. In at least one embodiment, the volume mechanism may be or include an actuation device.

The analysis unit 228, can determine the amount, concentration, and identify the drug, and/or medication. The analysis unit 228, in least one example, may include a spectrometer to allow for the identification and determination of the concentration of a received drug and/or fluid. The control system 232 may allow for the movement of a drug and/or medication from the receiving unit 222 to an audit system and/or storage 234, and/or a waste reservoir 236. In at least one example, a first portion of any drug and/or medication received by the receiving unit 222 is routed to the audit system and/or storage 234, and a second portion of any drug and/or medication received is routed to the waste reservoir 236. In at least one example, the analysis unit 228 can include a Raman Spectrometer to identify the drug and/or medication, and the drug and/or medication concentration, and/or a sensor for measuring volume. For example, the analysis unit 232 may be electrically coupled to a computing device 230 to determine the identification, concentration, and/or amount of a drug or medication received and/or analyzed by the receiving and/or analysis unit.

In some embodiments, the receiving unit 222 may be coupled to a fluid dispenser 224. The fluid dispenser 224 may allow for the receiving unit 222, to be rinsed and/or flushed with a diluent 226A, adulterant 226B, and/or flushing 226C fluid. The fluid dispenser 224, may include a fluid reservoir for storing one or more of the diluent 226A, adulterant 226B, and/or flushing 226C fluid. In other examples, the fluid dispenser 224 can be coupled to an input line or fluid line that allows for the diluent 226A, adulterant 226B, and/or flushing 226C fluid to be received from a main or building line, or external storage container.

The audit system and/or storage 234 can allow for the capture and/or storage of samples of the drug and/or medication received by the receiving unit 222. In at least one embodiment, the computing device 230 can receive signals from the analysis unit 228 that allow it to determine the identification, concentration, and amount of the drug and/or medication received. If the drug and/or medication does not match with one or more of the criteria set forth in the memory and/or storage of the computing device, and/or accessed from a remote memory and/or storage device then the computing device 230 can cause the control system 232 to transfer a portion of the drug and/or medication to the audit system and/or storage 234. In some examples, the entire amount of the drug and/or medication received may be transferred to the waste reservoir 236, and/or the audit system and/or storage 234.

FIG. 3 is an illustration of a medication lifecycle system 300 that may include a medication dispensing system and/or waste management system enclosure 340. The enclosure 340 may have openings 342A, 342B, 342C, 342D, 342E, 342F, 342G, and/or 342H (collectively 342). There may also be drawers 344A, 344B, and/or 344C (collectively 344), and/or storage lockers 346A, 346B, and/or 346C (collectively 346). The openings 342, drawers 344, and/or storage lockers 346 may allow for a drug and/or medication to be wasted and/or disposed of. In at least one embodiment, the drawers 344, and/or storage locker(s) 346 may be utilized for a medication dispensing system, and/or may be refrigerated. In at least one example, the openings may allow for different classes and/or concentrations of drugs and/or medications to be disposed of. For example, one sample may have a concentration of 1000 parts per million, while another sample has 10 parts per million. The separate openings 342, can allow for multiple analysis units (not illustrated) each focused on different concentration levels, and/or classes of drugs and/or medications. For example, a drug and/or medication may be in tablet form and/or powder form that have different analysis units to identify the drug and/or medication, concentration and/or amount of the drug and/or medication. An analysis unit (not illustrated) may be coupled to a computing device 330. The computing device 330 may also have user interface devices such as, but not limited to, a display 348, a keyboard 350, a mouse or touch pad 352, a card and/or near field communication reader 354, and/or a biometric device 358.

In at least one embodiment, the enclosure 340 may have a water or fluid line 356 that may lead to a water or fluid source, or an external water or fluid storage container. The water and/or fluid may allow for cooling and/ice to be generated for other drugs and/or medications. Additionally, the enclosure may have a power line (not illustrated) that allows for power to be sourced from an external power source such as but not limited to an AC line, and a DC battery.

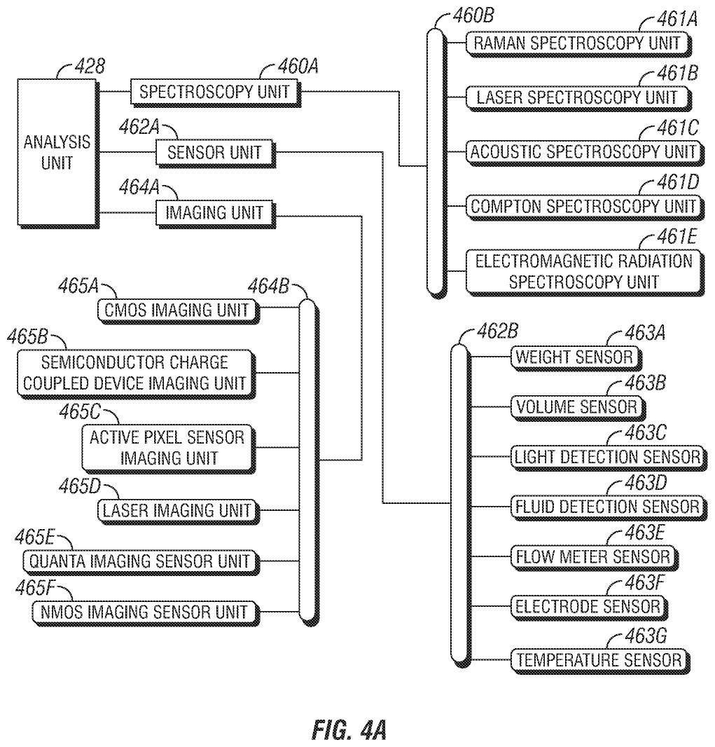

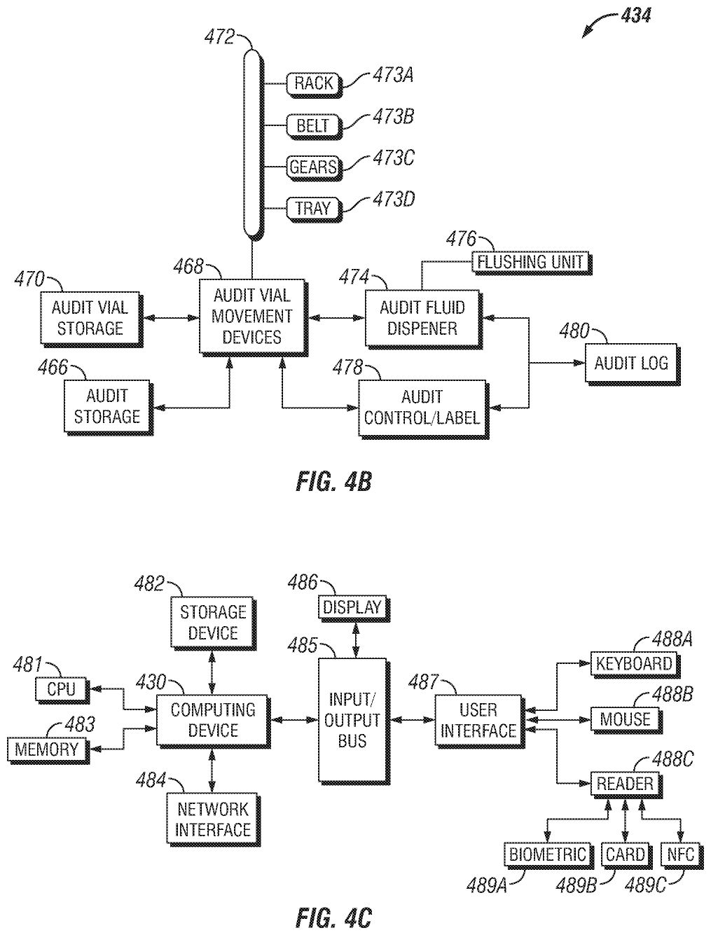

FIG. 4A is an illustration of an analysis unit 428. The analysis unit 428 may include a spectroscopy unit 460A, a sensor unit 462A, and/or an imaging unit 464A. These units 460A, 462A, and/or 464A can allow for the identification, concentration, and/or amount of a drug, medication, and/or other sample. The units may be coupled to a computing device, and/or input/output bus to interface with a computing device. In at least one example, the units may be wirelessly connected to a computing device.

A spectroscopy unit 460A, may include a connection and/or coupling bus 460B to allow multiple spectroscopy units to be utilized. For example, a Raman spectroscopy unit 461A, a laser spectroscopy unit 461B, an acoustic spectroscopy unit 461C, a Compton spectroscopy unit 461D, and electromagnetic radiation spectroscopy unit 461E.

A sensor unit 462A, may include a connection and/or coupling bus 462B to allow multiple sensor units to be utilized. For example, a weight sensor 463A, a volume sensor 463B, a light detection sensor 463C, a fluid detection sensor 463D, a flow meter sensor 463E, an electrode sensor 463F, and/or a temperature sensor 463G.

An imaging unit 464A, may include a connection and/or coupling bus 464B to allow multiple imaging units to be utilized. For example, a CMOS imaging unit 465A, a semiconductor charge coupled imaging unit 465B, an active pixel sensor imaging unit 465C, a laser imaging unit 465D, a quanta imaging sensor unit 465E, and/or a NMOS imaging unit 465F.

FIG. 4B is an illustration of an audit system and/or storage unit 434. The audit system and/or storage unit may include an audit vial storage 470. A vial (not illustrated) may be taken from the audit vial storage 470, with the audit vial movement devices 468. In at least one embodiment, a mechanical means 472 can provide movement for the vial to move through and/or about the audit system and/or storage unit 434. The mechanical means 472 may include a rack 473A, belts 473B, gears 473C, and/or trays 473D. In at least one example, the movement of the vial may include a combination of racks, belts, gears, and/or trays, as well as motors, power sources or systems, and other mechanical means of causing movement, rotation, and/or a change of location.

The vial may be filed through an audit fluid dispenser 474. In at least one example, an audit fluid dispenser 474 may be include in a control system (not illustrated) of a waste management system (not illustrated). The audit fluid dispenser 474 may be fluidly coupled to a waste management system (not illustrated). In at least one example, the audit fluid dispenser 474 can be coupled to a flushing unit 476. The flushing unity 476 can allow the audit fluid dispenser 474 to be cleaned before each dispensing of a drug and/or medication to be stored for audit purposes. This would be done to ensure pure and/or untainted samples. After the audit fluid dispenser 474 has dispensed a drug and/or medication, a signal may be sent to an audit log 480 and/or audit control and/or label system 478. The signal may include information regarding the user, the drug and/or medication, the time and/or date, and/or other pertinent information. The audit log 480 and/or audit control and/or label system 478 can allow for tracing and/or tracking of the audit vial. For example, the audit log 480 may include information about who was wasting drugs and/or medications, the time and/or date, location, information from a patient or staff database, what items were wasted with the drug and/or medication, what error and/or criteria triggered the audit, and/or the number of samples taken. Similar information may be included on an audit control and/or label system 478. Once a sample has been placed in the vial, and/or marked then it can be moved to an audit storage unit 466. In at least one example, the audit storage unit 466 may be secured with only a few individuals having access. An authorized user may have access to remove the vials held within and/or by an audit storage unit 466.

A computing device 430 may include and/or be coupled to many different devices and/or interfaces. Including a central processing unit 481, at least one storage device 482, at least one memory device 483, at least one network interface 484, and/or an input/output bus 485. In at least one example, an input/output bus 485 may be coupled to and/or included with the central processing unit (CPU) 481. A display 486, and/or other user interfaces 487 may be coupled to the input/output bus 485. In at least one example, the user interfaces 487 may include a keyboard 488A, a mouse 488B, and/or read/write devices 488C such as but not limited to, biometric 489A, card 489B, and/or NFC 489C, and/or other interface devices and/or systems capable of reading, scanning, and/or writing.

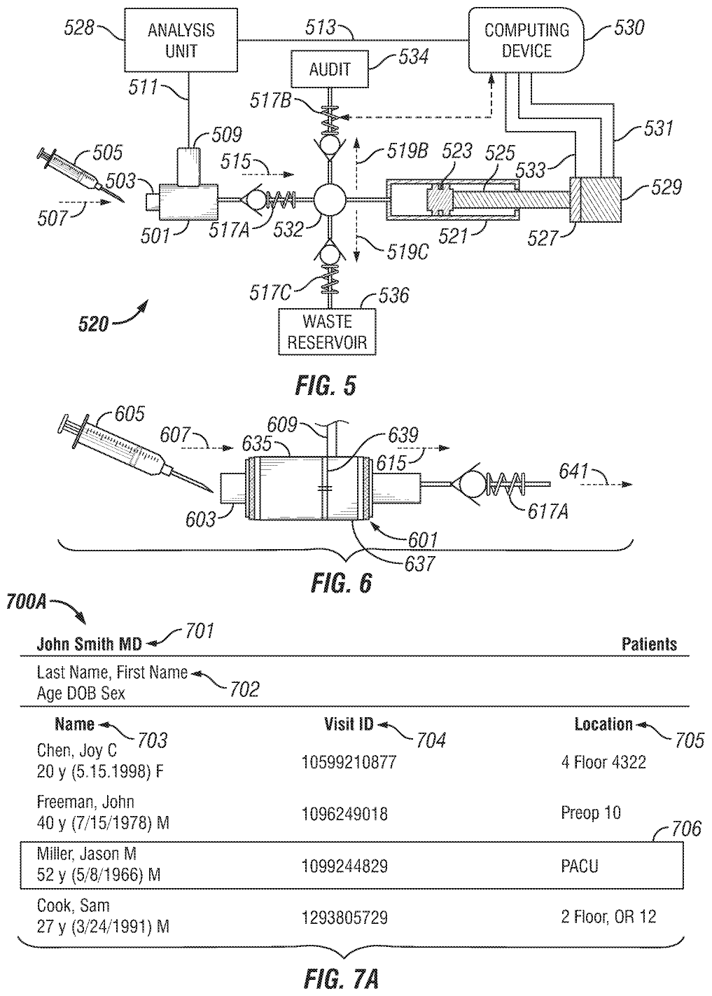

FIG. 5 is an illustration of a waste management system 520. The waste management system 520 can have a receiving unit 501. The receiving unit 501 can have a receiving point 503. The receiving point 503 in at least one example may be a Luer connection. The receiving point 503 can receiving the tip and/or point 507 of a syringe 505. In at least one example, the receiving point 503 may also receiving tablets, powders, and/or fluids. The syringe 505 may be any form of fluid transport device such as a vial, cup, bowl, or other containers.

The receiving unit 501 can be coupled to analysis unit 528 through an optical or sensor attachment device 509, and/or an optical and/or electrical connection 511. The analysis unit 528 can be electrically coupled 513 to a computing device 530. The electric coupling 513 may be a set of one or more outputs from the analysis unit 528. In at least one example, the receiving unit 501 can be cuvette holder and/or cuvette. A cuvette may be utilized to create a known volume to allow for a proper analysis of a drug and/or medication. One example may be a cuvette with a volume equivalent to 50 mm³. As a drug, and/or medication is received by the receiving unity 501 it can be transferred to an audit system and/or storage 534 or a waste reservoir 536. A fluid such as, but not limited to a drug and/or medication can be pulled and/or siphoned 515 from the receiving unit with a volume mechanism 521, the volume mechanism 521 may be defined as a barrel with a plunger head 523, plunger rod 525 coupled to an encoder 527, and/or a stepper motor 529. The stepper motor 529 can be coupled to the computing device 530 through an electrical connection 531. The encoder 527 can be coupled 533 to the computing device 530 and/or the stepper motor 529. The encode 527 in at least one embodiment, may be electrically coupled 533 to the computing device 530. In at least one example, the analysis unit 528 can be a Raman spectrometer.

The volume mechanism 521 can allow for the movement of a drug and/or medication through a control system. The control system may include at least one control valve 517A, 517B and/or 517C (collectively 517). The control system may allow for a drug and/or medication to flow 515 through a control valve 517A, and then be distributed or portioning 519A and/or 519B the drug and/or medication through control valve 517B, and/or 517C. In at least one embodiment the control valves 517 can be controlled and/or operated by the computing device 530. The control valves 517 may also be check valves to prevent back flow. The volume mechanism may include a barrel and plunger/piston attached to linear actuator and/or encoder. In at least one example, a waste reservoir can be secured to make wasted drugs and/or medications irretrievable. A syringe may have a Luer connector, Luer lock connector, and/or a needle that may be coupled and/or locked with the receiving point that may also have a slot or opening, and/or a Luer connector or Luer lock connector. In at least one example, the volume mechanism can draw the contents of the syringe into the receiving unit, and/or an analysis portion of the receiving unit. The receiving unit, may have a check or control valve configured for one way flow, to prevent a portion of the fluid from returning to the syringe and/or receiving unit. As the volume mechanism moves and/or a plunger barrel combination or other volume changing device moves, via a motor or other mechanical movement method. Some of the other mechanical movement methods could include a linear plunger and/or piston inside a barrel driven by a linear actuator connected to an encoder, a linear drive mechanism, stepper motor, and/or DC motor and screw, each of which may also be paired with an encoder. Depending on accuracy and life time requirements a linear potentiometer could be used to determine distance traveled. In at least one embodiment, an encoder, or a stepper motor may be utilized to ensure that the movement of the plunger or other volume changing device is properly recorded. For example, with a barrel plunger combination, the volume and/or amount of drugs and/or medications would be the distance the plunger has traveled multiplied by the cross section of the barrel.

FIG. 6 is an illustration of the receiving unit 601. The receiving unit 601 can include a cuvette holder 635, and/or a cuvette 637. The cuvette 637 may be sized to receive a proportionally sized amount of drug and/or medication to allow for analysis. The cuvette may also be coupled to an analysis unit through a sensor, electrical, and/or optical attachment 609. The attachment may include coupling to a sensor within 639 the cuvette and/or between the cuvette holder 635 and the cuvette 637. In at least one embodiment, the cuvette holder 635, and cuvette 637 are a receiving unit 601, with the cuvette 637 being an analysis section and the cuvette holder 635 being a holding section. The receiving unit 601 can have a receiving point 603. The receiving point 603 in at least one example may be a Luer connection. The receiving point 603 and/or the sensor within 639 may include an anti-siphon valve. The receiving point 603 can receiving the tip and/or point 607 of a syringe 605. In at least one example, the receiving point 603 may also receiving tablets, powders, and/or fluids. The drug and/or medication may flow 615 from the receiving unit 601 to a control valve 617. In at least one embodiment, the control valve 617 can be controlled by a computing device (not illustrated) to allow or not allow a drug and/or medication to flow to a control system or a control system flow 641.

A syringe will typically hold a minimum volume of 0.25 ml. In at least one example, the cuvette 637 may have an internal volume of 50 mm³ (μL). In an example, with a drug and/or medication in the amount described above, the internal cuvette volume will be diluted 5 times after a previous sample. The amount of previous sample after 5 dilutions would be 3.125% allowing multiple samples to be taken in quick succession as the cuvette 637 cannot hold the entire amount wasted from a syringe 605, allowing the cuvette holder 635 to hold the remaining portion of the sample.

In at least one example, the longer the sample time the higher photo signal and/or counts for a given molecule and/or sample. Additionally, the higher the concentration the less time required to analyze the sample. The ability and/or time to detect a drug and/or medication from a sample, can be less than the time to quantify and/or identify the sample. The cuvette 637 may be customized to create a discrete sample size for any drug and/or medication.

In some examples, the waste management system and/or apparatus may notify a pharmacy if a diversion event is identified. A diversion event could be when a sample has been tainted and/or adulterated beyond recognition, e.g., someone putting two or more drugs and/or medication at full strength together as a single sample. The waste management system and/or apparatus can send a report of the diversion event to a pharmacy, hospital, or other administration system or service. In some examples the waste management system and/or apparatus may generated and/or provide a report regarding the reason to suspect diversion—wrong drug, wrong concentration or amount. In at least one example, the waste management system and/or apparatus can continue to accept and/or analyze additional samples as the sample that triggers the diversion warning and/or alert may be moved and/or transferred to an audit system and/or vial.

In at least one example, the pharmacy may have the option to receive real time notifications of suspected diversion and to stop device operations remotely. The device would enter standby mode and no longer accept additional samples. The cuvette/sample would be retrieved, and a new cuvette inserted and device operation would be restarted. In other embodiments, the pharmacy could trigger the movement and/or transfer of the sample to an audit system and/or vial upon review of an alert from the waste management system. The cuvette and/or cuvette holder may have anti-siphon valves located at one or more of the openings to preserve the integrity of the sample. In at least one embodiment, the anti-siphon valves may be incorporated within a male and/or female Luerk connection. Additionally, the audit system and/or vials, and/or control valves may have anti-siphon valves.

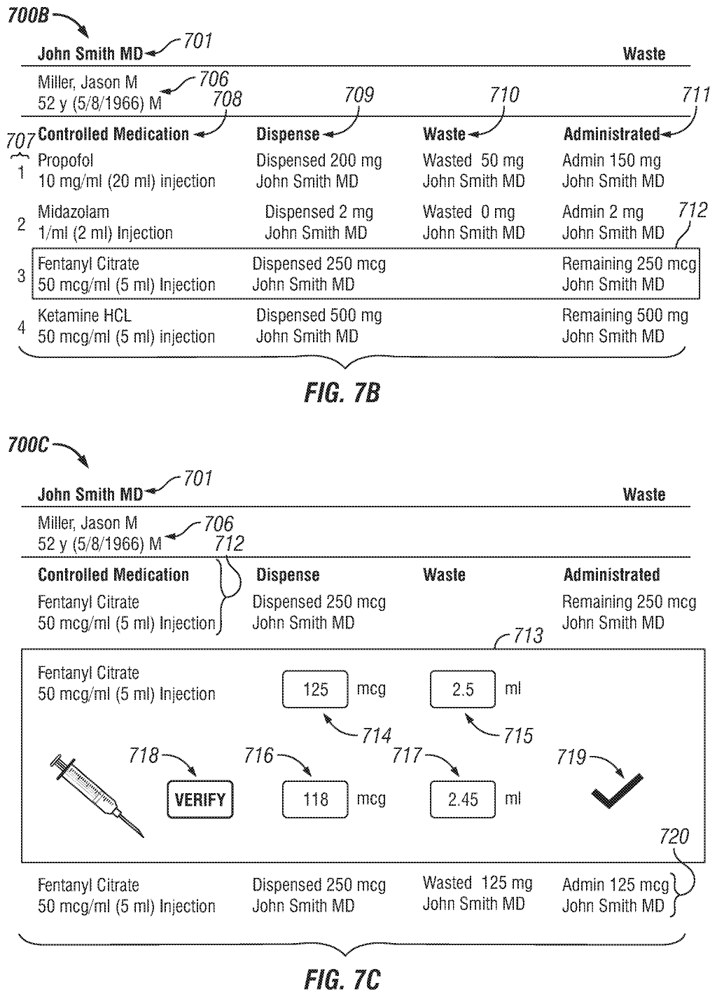

FIG. 7A is an illustration of a waste management system display screen 700A. In at least one example the waste management system display screen 700A is provided and/or displayed to a user through the waste management system and/or a computing device coupled to the waste management system. The waste management display screen 700A can allow for user and/or patient information to be provided and/or displayed. User information 701 can be provided and/or displayed, and/or may include the name of the user, their position, and/or ID credentials such as, but not limited to employee number, identification method, and/or login location (could be used to identify an imposter or a mishandling). Patient information 702 may also be provided and/or displayed. The patient information may include name, age, date of birth, sex, location, patient identifier, visit identification, and/or other criteria. In at least one example, the patent name 703, visit ID 704, and/or location 705 can be provided to allow a user to correctly select the patient to whom the drugs and/or medications are assigned. A user may select 706 one of the patient from a list and/or group and/or set of patients. In at least one example, the list, group, and/or set of patients displayed are organized from those assigned to the user, and/or the user's supervisor.

For example, a user may log into the waste management system using password and/or additional identification methods such as a fingerprint ID. If the user is authenticated and/or allowed into the system a patient list can be displayed and/or provided to the user. The user can select a patient name from patient list. In at least one example, once a patient is selected a drug and/or medication information page for that patient may be displayed and/or provided to the user.

FIG. 7B is an illustration of a waste management system display screen 700B. The display screen 700B can include user information, patient information, and/or drug and/or medication information. In at least one example, a user's information 701 may be included on the display screen 700B to allow a user to verify they are the correct user for the drugs and/or medication. Some examples, may allow for the transfer of the drugs and/or medication to another user, if approved by that user. Information about a selected patient 706 may be presented along with information regarding the drugs and/or medications. For example, the waste management system may indicate that a drug and/or medication 708, should be wasted in an assigned order 707 to ensure proper analysis. The drug and/or medication 708, can have the amount dispensed 709 presented and/or displayed to allow a user to make any notes of an error. The waste management system may also calculate how much was administered 711 and/or wasted 710 based on the analysis and/or amount of the drug and/or medication dispensed 709. In at least one example, the waste management system may highlight and/or select 712 a drug or medication to be wasted based upon the known concentrations and/or amount to be wasted.

For example, a user (not illustrated) may select the drug and/or medication to be wasted from the waste management system display screen 700B. The drugs and/or medications may be displayed and/or provided to the user in a list and/or ordered presentation. The list and/or ordered presentation can be based on the control level of the drug and/or medication. E.g., a narcotic may be listed first in the list because it must be tracked and/or recorded each time it is handled. Another example of the list could be based on the concentration level of the drug and/or medication. In at least one example, the administrated amount of a drug and/or medication is determined by subtracting the waste amount from the dispensed amount. The calculated administrated amount should correlate with what the user has documented in the medical record of the patient selected within a specified and/or programmed error factor, such as 0% to 10%.

Of note, the waste management system does not detect drug diversion at the point of administration. For instance, midazolam is documented as being administered in entirety to the patient. The provider, doctor, and/or nurse could swap the midazolam with saline, claimed to have administered it all, and avert detection by the waste system. However, a measurement device and/or drug or medication monitoring device could be adapted to allow for point of administration interaction with the waste management system. This would allow the administrated amount of a drug and/or medication to be determined has opposed to calculated.

FIG. 7C is an illustration of a waste management display screen 700C. The display screen 700C can provide information regarding a user, a patient, and/or drug and/or medication. For example, a user's information 701 may be included to allow for verification that the proper user is in fact the user assigned to the patient and/or drugs and/or medication. A patient's information 706, and/or the information for a selected patient may be provided to allow a user to verify the patient to which the drugs and/or medication may be assigned. The selected drug and/or medication from FIG. 7B may be presented and/or displayed in more detail. For example, the selected drug and/or medication 712, may be Fentanyl citrate.

A details section 713 can allow a user to know the concentration 714, and/or amount 715 of the drug and/or medication that was dispensed. A measured and/or calculated concentration 716, and/or amount 717 of a drug may be provided and/or displayed after and/or during the wasting. In at least one example, a user may request verification 718 that the drug and/or medication has been properly wasted. An alert and/or indicator 719 can let the user know that they have properly wasted the correct amount, concentration, and/or drug and/or medication. An updated drug and/or medication section 720 can allow the user to verify that the proper information was properly saved and/or stored. In some examples, a user must enter the amount they plan to waste 716 and/or 717. For example, the user may entered a waste amount of 125 mcg 716 and the corresponding volume of 2.5 ml 717. Some examples, may allow the user to input one of the concentration 716 and/or the volume 717 to be wasted. The user (not illustrated) may select the sample to be verified 718. Based on the analysis performed by the waste management system, the user may be provided and/or an indicator 719 to indicate the result of the verification. In at least one embodiment, the indicator 719 may only indicate a positive result. In some examples, the indicator 719 can provide a positive and/or a negative verification status.

If there is a significant mismatch between the amounts entered by the user (not illustrated) and the sample analyzed, a notification may be immediately sent to the institution's pharmacy and/or facility's administration with a report documenting the discrepancy and alerting the pharmacy to a possible diversion event. The report may include dispensed and wasted amounts as entered by the user as well as Raman spectrum for the sample, calculation of concentration based on the analysis, and volume of sample. The waste sample will be preserved for extraction from the device. This would allow additional toxicology testing.

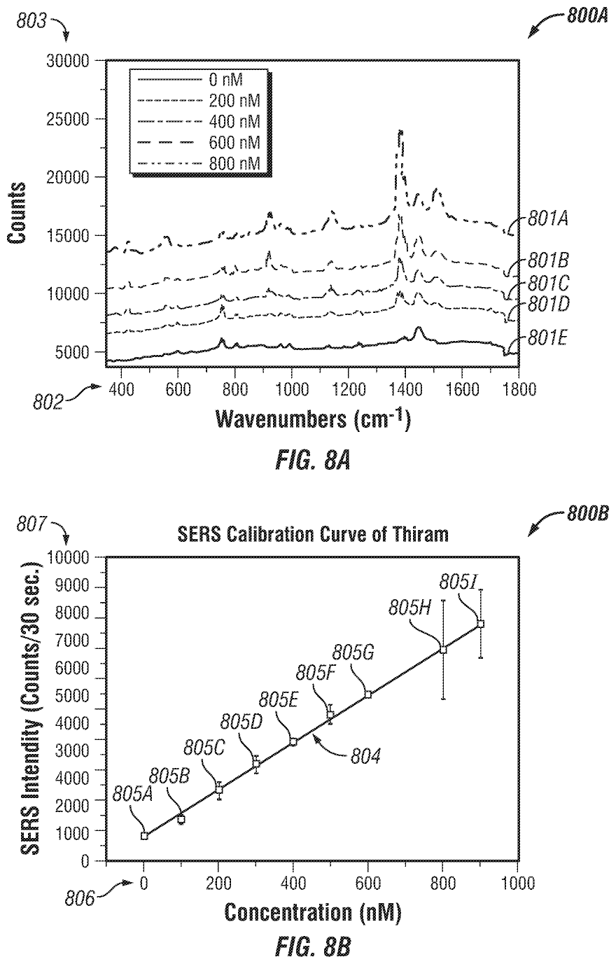

FIG. 8A is an illustration of a Raman spectrometry count curves 800A. The count curves 801A, 801B, 801C, 801D, and/or 801E can allow for a measurement of a concentration of a drug and/or medication or other sample. The wave and/or frequency (other spectrometer systems) 802 and the count values 803 can allow for concentration curves to be generated for various drugs and/or medications. The Raman spectra is unique for each drug and/or medication being analyzed. A database of known Raman spectra containing each drug and/or medication, e.g., fentanyl, morphine, Propofol, etc., may be created, accessed, and/or updated.

FIG. 8B is an illustration of Raman spectrometer concentration curves 800B. The concentration curve 804 may be created based on an analysis be a Raman or other spectrometer unit. The concentration curve 804 can also have various points 805A, 805B, 805C, 805D, 805E, 805F, 805G, 805H and/or 8051 (collectively 805). The points can allow for a drug and/or medication to be calibrated 806 based on various concentrations and/or count measurements 807. These points 805 can also include a window of approved concentrations that can be reviewed.

The concentration curve equation and/or line will be determined for each medication in the database of known Raman spectra. In at least one example, the concentration curve is the photon count at various concentration of medication. In some examples, a least squares regression analysis will determine concentration curve equation for each drug and/or medication in the database of known Raman spectra. For each drug and/or medication being wasted that has an unknown Raman spectra, the sample with be analyzed and/or compared to the database of known Raman Spectra to identify the drug and/or medication. Based on the strength of Raman signals in the sample the concentration will be calculated and/or matched using the corresponding concentration curve equation and/or line.

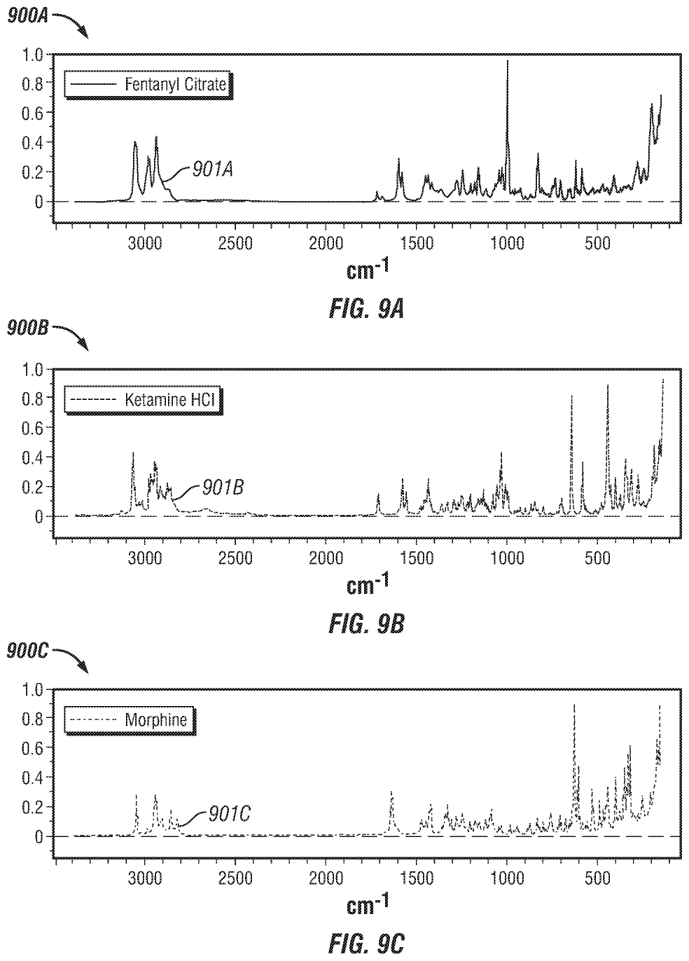

FIG. 9A is an illustration of a Raman spectrometer result 900A for Fentanyl Citrate. The result allows for the response 901A of the counts to be displayed and/or provided. In at least one example, the result can be saved for later identification of other similar drugs and/or medications.

FIG. 9B is an illustration of a Raman spectrometer result 900B for Ketamine HCl. The result allows for the response 901B of the counts to be displayed and/or provided. In at least one example, the result can be saved for later identification of other similar drugs and/or medications.

FIG. 9C is an illustration of a Raman spectrometer result 900C for Morphine. The result allows for the response 901C of the counts to be displayed and/or provided. In at least one example, the result can be saved for later identification of other similar drugs and/or medications.

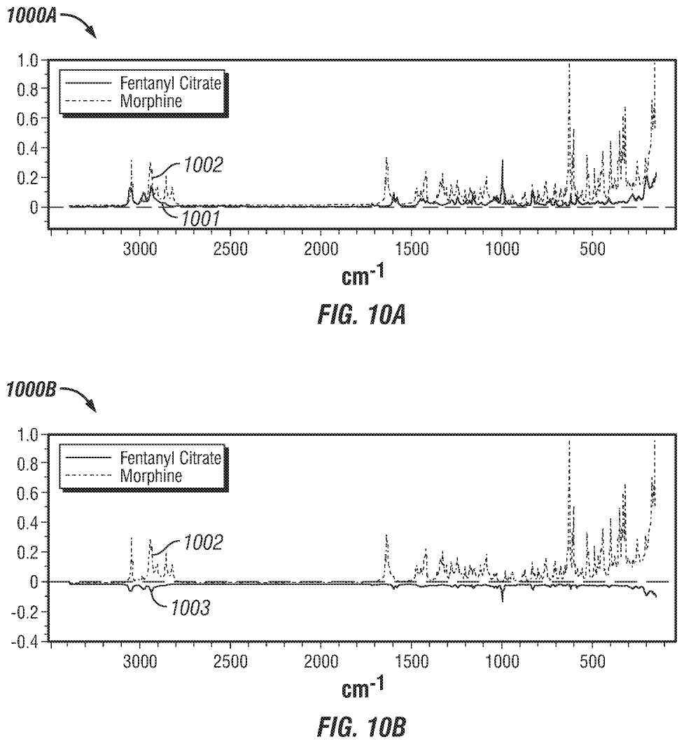

FIG. 10A is an illustration of a sample 1000A that includes two different drugs and/or medications. A first drug and/or medication 1001, and a second drug and/or medication 1002. The first drug and/or medication 1001 may be left over from a previous sample, and/or an analysis unit that was not cleaned.

FIG. 10B is an illustration of a sample 1000B that has had the first drug and/or medication 1001, subtracted and/or removed 1003 from the second drug and/or medication 1002. It should be noted that portions of the present disclosure may be implemented through a computer readable medium, and/or a website, site, webpage, page, web script, or script. The website, site, webpage, page, web script, or script could include individual pages, scripts, sites, or applications, or a group of pages, scripts, sites, or applications. For example, Google® can be an individual site or page depending on the reference or view, or could be a group of pages or sites.

Additionally, it would be understood that a browser or program could be implemented on a mobile device, such as, a phone, a mobile phone, a cell phone, a computer, a tablet, a laptop, a mobile computer, a personal digital assistant (“PDA”), a processor, a microprocessor, a micro controller, or other devices or electronic systems capable of connecting to a user interface and/or display system such as a computing device.

The present disclosure may also comprise a computing device that can include any of an application specific integrated circuit (ASIC), a microprocessor, a microcontroller, a digital signal processor (DSP), a field-programmable gate array (FPGA), or equivalent discrete or integrated logic circuitry such as but not limited to a Central Processing Unit. In at least one embodiment, the central processor unit could include an ASIC, microprocessor, microcontroller, DSP, FPGA, or other discrete or integrated logic circuits. In some examples, the system may include multiple components, such as any combination of one or more microprocessors, one or more microcontrollers, one or more DSPs, one or more ASICs, or one or more FPGAs. It would also be understood that multiples of the circuits, processors, or controllers could be used in combination or in tandem, or multithreading.

The components of the present disclosure may include any discrete and/or integrated electronic circuit components that implement analog and/or digital circuits capable of producing the functions attributed to the systems, methods, or modules herein. For example, the components may include analog circuits, e.g., amplification circuits, filtering circuits, and/or other signal conditioning circuits. The components may also include digital circuits, e.g., combinational or sequential logic circuits, memory devices, etc. Furthermore, the modules may comprise memory and/or storage devices that may include computer-readable instructions that, when executed cause the modules to perform various functions attributed to the modules herein.

Memory may include any volatile, non-volatile, magnetic, or electrical media, such as a random access memory (RAM), dynamic random access memory (DRAM), static random access memory (SRAM), read-only memory (ROM), non-volatile RAM (NVRAM), electrically-erasable programmable ROM (EEPROM), flash memory, hard disks, or any other digital media. Additionally, there may also be a tangible non-transitory computer readable medium that contains machine instructions, such as, a (portable or internally installed) hard drive disc, a flash drive, a compact disc, a DVD, a zip drive, a floppy disc, optical medium, magnetic medium, solid state medium, or any other number of possible drives or discs, that are executed by the internal logic of a computing device. It would be understood that the tangible non-transitory computer readable medium could also be considered a form of memory, storage device, or storage media.

While this disclosure has been particularly shown and described with reference to preferred embodiments, it will be understood by those skilled in the art that various changes in form and detail may be made therein without departing from the spirit and scope of the invention. The inventors expect skilled artisans to employ such variations as appropriate, and the inventors intend the invention to be practiced otherwise than as specifically described herein. Accordingly, this disclosure includes all modifications and equivalents of the subject matter recited in the claims appended hereto as permitted by applicable law. Moreover, any combination of the above-described elements in all possible variations thereof is encompassed by the disclosure unless otherwise indicated herein or otherwise clearly contradicted by context.

While various embodiments in accordance with the principles disclosed herein have been described above, it should be understood that they have been presented by way of example only, and not limitation. Thus, the breadth and scope of this disclosure should not be limited by any of the above-described exemplary embodiments, but should be defined only in accordance with any claims and their equivalents issuing from this disclosure. Furthermore, the above advantages and features are provided in described embodiments, but shall not limit the application of such issued claims to processes and structures accomplishing any or all of the above advantages.

Additionally, the section headings herein are provided for consistency with the suggestions under 37 C.F.R. 1.77 or otherwise to provide organizational cues. These headings shall not limit or characterize the invention(s) set out in any claims that may issue from this disclosure. Specifically, and by way of example, although the headings refer to a “Technical Field,” the claims should not be limited by the language chosen under this heading to describe the so-called field. Further, a description of a technology as background information is not to be construed as an admission that certain technology is prior art to any embodiment(s) in this disclosure. Neither is the “Brief Summary” to be considered as a characterization of the embodiment(s) set forth in issued claims. Furthermore, any reference in this disclosure to “invention” in the singular should not be used to argue that there is only a single point of novelty in this disclosure. Multiple embodiments may be set forth according to the limitations of the multiple claims issuing from this disclosure, and such claims accordingly define the embodiment(s), and their equivalents, that are protected thereby. In all instances, the scope of such claims shall be considered on their own merits in light of this disclosure, but should not be constrained by the headings set forth herein.

We claim:

1. A waste management system comprising:
a receiving unit having at least one anti-siphon valve, a holding section, and an analysis section;
an analysis unit coupled to the analysis section of the receiving unit, and configured to analyze a fluid for identification, concentration, and amount;
at least two control valves fluidly coupled to the receiving unit;
a waste reservoir fluidly coupled to one or more of the at least two control valves; and
a computing device having at least one storage device, at least one memory unit, one or more central processing units, a display, an input/output bus, and at least one user interface device coupled to the input/output bus;
wherein the computing device is coupled to the analysis unit, and the at least two control valves.

2. The waste management system of claim 1, further comprising at least one actuation device coupled to one or more of the at least two control valves.

3. The waste management system of claim 2, wherein the at least one actuation device further comprises a plunger, a barrel, and a stepper motor coupled to the plunger.

4. The waste management system if claim 1, wherein the analysis unit further comprises a spectroscopy unit.

5. The waste management system of claim 4, wherein the spectroscopy unit further comprises one or more spectroscope unit chosen from a group consisting of a raman spectroscopy unit, a laser spectroscopy unit, an acoustic spectroscopy unit, a Compton spectroscopy unit, and electromagnetic radiation spectroscopy unit.

6. The waste management system of claim 1, wherein the analysis unit further comprises at least one sensor.

7. The waste management system of claim 6, wherein the at least one sensor is chosen from a group consisting of weight sensors, volume sensors, light detection sensors, fluid detection sensors, electrodes.

8. The waste management system of claim 1, wherein the analysis unit further comprises at least one imaging unit.

9. The waste management system of claim 8, wherein the at least one imaging unit is chosen from a group consisting of a CMOS imaging unit, a semiconductor charge-coupled device imaging unit, an active pixel sensor imaging unit, laser imaging unit, Quanta imaging sensor unit, and NMOS imaging unit.

10. The waste management system of claim 1, wherein the waste reservoir further comprises an adulterant to be mixed with the fluid.

11. A waste management apparatus comprising:
a receiving unit with at least one anti-siphon valve, and having a first side configured for receiving a fluid transport device;
a holding section of the receiving unit fluidly coupled to the first side of the receiving unit;
an analysis section of the receiving unit fluidly coupled to the holding section and a second side of the receiving unit;
an analysis unit coupled to the analysis section;
at least two control valves fluidly coupled to the analysis section;
a waste reservoir fluidly coupled to one or more of the at last two control valves; and
at least one computing device for controlling and receiving signals from the analysis unit and the at least two control valves.

12. The waste management apparatus of claim 11, wherein the holding section has a first side fluidly coupled to at least one anti-siphon values, and a second side fluidly coupled to the analysis section.

13. The waste management apparatus of claim 11, wherein the analysis section has a first side fluidly coupled to at least one analysis section anti-siphon valves, and a second side fluidly coupled to one or more of the at least two control valves.

14. The waste management apparatus of claim 13, wherein the analysis section is fluidly coupled to the holding section through one or more of the at least one analysis section anti-siphon valves.

15. The waste management apparatus of claim 11, wherein the analysis unit is mechanically coupled to the analysis section of the receiving unit.

16. The waste management apparatus of claim 11, wherein the analysis unit is optically coupled to the analysis section of the receiving unit.

17. The waste management apparatus of claim 11, wherein the computing device controls the at least two control valves based on a set of outputs from the analysis unit.

18. The waste management apparatus of claim 11, wherein a portion of a fluid is directed to an audit unit.

19. The waste management apparatus of claim 11, further comprises an audit unit fluidly coupled to the at least two control valves.

20. A waste management method comprising:
receiving a fluid via a receiving unit;
transferring the fluid to an analysis section of the receiving unit;
analyzing the fluid for identification, concentration, and amount with an analysis unit wherein the analysis unit is electrically coupled to a computing device; and
directing the fluid to a waste reservoir that is fluidly coupled to the analysis unit.

21. The waste management method of claim 20, wherein the receiving unit has a holding section that is fluidly coupled to the analysis section.

22. The waste management method of claim 20, wherein the analysis unit further comprises scanning the fluid.

23. The waste management method of claim 20, wherein the analysis unit further comprises imaging the fluid.

24. The waste management method of claim 20, wherein the analysis unit further comprises weighing the fluid.

25. The waste management method of claim 20, wherein the analysis unit further comprise metering the fluid.

26. The waste management method of claim 20, further comprising moving the fluid from the analysis section with an actuator.

27. The waste management method of claim 20, wherein the directing step further comprises directing a first portion of the fluid to an audit storage unit and a second portion of the fluid to the waste reservoir.
